(19) United States
(12) Patent Application Publication
(10) Pub. No.: US 2012/0004619 A1
(43) Pub. Date: Jan. 5, 2012

(54) NEEDLE PROTECTIVE DEVICE

(76) Inventors: JOHN D. STEPHENS, Alpharetta, GA (US); John Muri, Laguna Niguel, CA (US); Ken Garry, El Cajon, CA (US); Ron Bark, San Diego, CA (US); John Detloff, San Diego, CA (US); James O'Conner, San Diego, CA (US); Gary Brown, Jupiter, FL (US)

(21) Appl. No.: 13/077,625

(22) Filed: Mar. 31, 2011

Related U.S. Application Data
(60) Provisional application No. 61/319,644, filed on Mar. 31, 2010.
(60) Provisional application No. 61/443,553, filed on Feb. 16, 2011.

(51) Int. Cl. A61M 5/32

(52) U.S. Cl. 604/198

(57) ABSTRACT

A needle protective device is provided for reducing inadvertent needle sticks. The needle protective device includes a tubular member that is attached to a base cap. A spring and a shield are positioned in the base cap to selectively cover an access hole of the chamber so that after use of the needle, a needle tip and blood-borne pathogens on the needle are encased within the base cap. The device is selectively pre-activated and is configured such that when activated, the needle tip is contained in the base cap and the device becomes un-usable.

### CROSS-REFERENCE TO RELATED APPLICATIONS

This application claims the benefit of U.S. Application No. 61/319,644, filed Mar. 31, 2010, and U.S. Application No. 61/443,553, filed Feb. 16, 2011; each of which is hereby incorporated herein by reference in its entirety.

### FIELD OF THE INVENTION

This invention relates generally to the field of hypodermic needles and Huber Infusion Sets, and more specifically, a needle protective device to reduce inadvertent needle stick incidents and help provide microbial protection at port wound sites.

### BACKGROUND OF THE INVENTION

Infectious diseases can be transmitted to medical personnel and others by way of inadvertent needle sticks. Needle stick injuries occur frequently, most often between the time the medication is injected into the patient and the time the syringe or infusion sets are disposed of Injuries occur before, during and after the clinical process. Needle stick injuries after use of the needle have been reduced with current needle protective devices, but needle stick injuries and microbial protection remain unaddressed during the clinical process.

Long term infusion of a patient through port access can cause microbial contamination at a wound site. Wound dressing are designed to intimately contact the wound sites as primary dressing to permit passage of fluids. It is therefore desirable to provide a needle protective device that reduces the occurrence of microbial contamination of the wound site of the patient. It is also desirable to provide a needle protective device to reduce the infection risk factors and number of needle stick incidents during the complete clinical process. It is also desirable to provide a Huber Infusion Set that provides protection at point of contact with skin to reduce occurrences of microbial contamination.

### SUMMARY OF THE INVENTION

According to various embodiments, a needle protective device is provided for reducing inadvertent needle sticks in infusion clinical situations as well as providing infectious control at port wound sites. In one embodiment, the needle protective device comprises a needle, a tubular member and a base cap. In one aspect, the needle protective device can further comprise a Huber infusion set. In another aspect, the needle can comprise a Huber needle.

In one aspect, the tubular member is formed from a resilient material that provides a protective spring encasement feature such that, when the device is in use, the needle tip (containing bloodborn pathogens) can be offset and isolated within the base cap. In another aspect, the device can be pre-activated prior to the clinical procedure in a hygienic manner, reducing the exposure of a user to an exposed tip of the needle throughout the clinical procedure of insertion of the needle into a patient. In another aspect, it is contemplated that upon removal of the needle from the patient, the needle tip can be passively encased in the base cap.

In another aspect, the base cap can comprise a spring and/or a spring cover. In this aspect, the spring and/or the spring cover can be configured to selectively restrict access of the needle tip to an access hole of the base cap, thereby reducing inadvertent needle sticks to a user.

In still another aspect, the needle protective device can further comprise a protective dressing that provides passage of oxygen and fluids at the wound site. In one aspect, the protective dressing can comprise metallic silver and/or silver oxides that can release a sustained level of silver ions that provide an antimicrobial barrier at the wound site. In another aspect, the antimicrobial barrier can protect the wound site against microbial contamination with materials suitable for providing or otherwise delivering a desired level of antimicrobial ions to a wound site directly or indirectly.

In one aspect, the needle-Huber protective device can be a passive protective device provided for reducing inadvertent needle sticks in infusion clinical situations as well as providing infectious control at wound port sites. In one exemplary aspect, the needle-Huber protective device can comprise a tubular member and a base cap. In this aspect, the base cap can comprise a protective shield and spring locking feature and/or other base cap covering mechanism that is configured to isolate the needle tip (and any pathogens on the needle tip) within a chamber basin of the base cap. In another aspect, the base cap can be attached via the tubular member to a Huber needle hub and wing component.

In one aspect, the device can be pre-loaded (activated) in a hygienic aseptic manner prior to the clinical procedure of insertion of the Huber infusion set into a vascular access port of a patient and removal therefrom. Upon removal of the device from the patient, passive encasement of the needle tip within the tubular member and base cap reduce the exposure of a user to an exposed needle tip. Advantageously, the post activated encasement of the needle tip within the tubular member and the base cap can help ensure the user that the safety feature is activated passively and remains in a protective condition throughout the clinical and disposal process.

The base cap can be formed from a rigid material and can define the chamber basin. In one aspect, the chamber basin defines an access hole through which the needle of the device can be passed. In another aspect, a portion of the protective shield spring locking feature can be mounted around and/or within a rigid material of the base cap, such as a shield spring support column. As exemplarily configured, when the needle extends through the access hole, the needle can rest against the pre-activated shield spring, which prevents the shield from covering the access hole.

Prior to safety activation, the needle tip can extend through the access hole and out of the base cap. In one aspect, the needle tip can be protected using a conventional flexible or hinged material. In another aspect, it is contemplated that one or multiple geometrically shaped stop ridges or tabs can be formed within the chamber basin of the base cap from substantially rigid material to ensure that the shield spring can rest in a closed (locked) and/or open (unlocked) position as desired. In still another aspect, the geometrically shaped stop ridges can be configured to prevent movement of the shield and spring beyond a predetermined position.

In one aspect, upon activation of the device, the access hole of the base cap can be automatically covered by the pre-activated shield-spring. In this aspect, the tip of the needle can be moved into and within the chamber basin above and/or away from the access hole by means of the resilient force stored within the tubular member when compressed and an activation force provided by the user. In another aspect, the activation of the device passively activates the shield, thereby covering the access hole of the base cap and locking and/or connecting the shield to the geometrically shaped stop ridges. This can prevent movement of the shield and/or spring while providing a means for locked safety features, as well as preventing the reuse or reactivation of the device.

Additional advantages of the invention will be set forth in part in the description that follows, and in part will be obvious from the description, or can be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

### BRIEF DESCRIPTION OF THE DRAWINGS

In the accompanying drawings which illustrate by way of example preferred embodiments of the invention:

FIG. 1 is a side elevational view of a needle protective device showing a base cap and a needle assembly mounted to a tubular member in an uncompressed post-activated position, according to one aspect.

FIG. 2 is a side cross-sectional view of the needle protective device of FIG. 1 showing the tubular member in a compressed, pre-activated position.

FIG. 3 is a perspective view of the needle protective device of FIG. 1 showing the tubular member in the post-activated position.

FIG. 4 is a top plan view of the of the needle protective device of FIG. 3.

FIG. 5 is bottom view of the needle protective device of FIG. 1 showing the tubular member in the compressed, pre-activated position.

FIG. 6 is a side elevational view of the needle protective device of FIG. 5 showing the tubular member in the compressed, pre-activated position.

FIG. 7 is side elevational view of the needle protective device of FIG. 3, showing the tubular member in the uncompressed post-activated position.

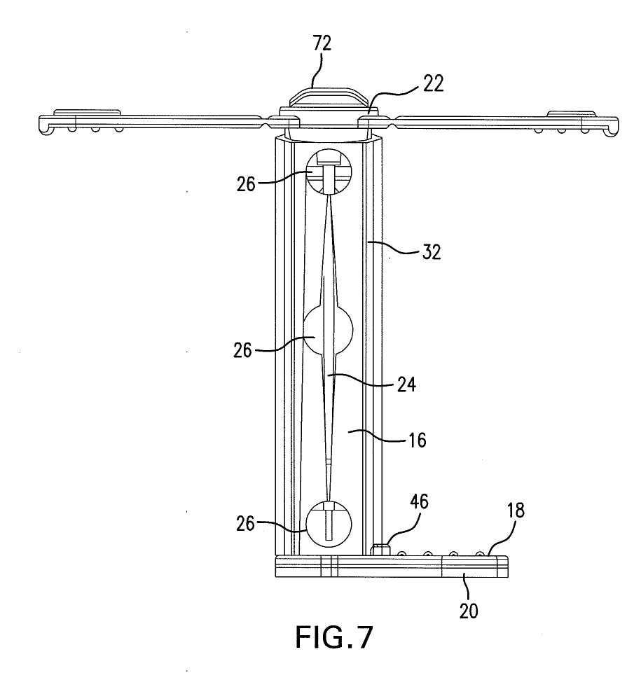

FIG. 8 is a perspective view of the base cap of the needle protective device of FIG. 1, according to one aspect, showing a pre-activated torsion spring and shield.

FIG. 9 is a perspective view of the base cap of the needle protective device of FIG. 1, according to one aspect.

FIG. 10 is a side elevational view of the needle protective device of FIG. 2, showing a tubular member in the compressed position.

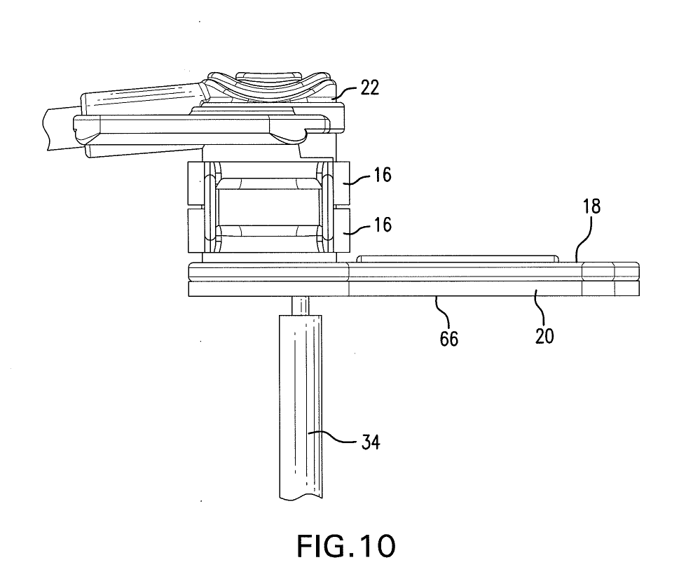

FIG. 11 is a perspective view of the base cap of the needle protective device of FIG. 1, according to one aspect, showing a post-activated spring and shield with the shield in a closed position.

FIG. 12 is an enlarged perspective view of one aspect of the base cap of the needle protective device of FIG. 1, according to one aspect, showing a post-activated spring and shield with the shield in a closed position.

FIG. 13 is an enlarged perspective view of one aspect of the base cap of the needle protective device of FIG. 1, according to one aspect, showing a plurality of locking members and slots.

FIG. 14 is a top view of two needle protective devices that are shaped or otherwise configured to complementarily mate with each other to allow multiple infusions at one port site.

FIG. 15 is a plan view of a portion of a needle-Huber protective device showing a base cap and a Huber-needle extending through an access hole of the device, according to one aspect.

FIG. 16 is a plan view of a portion of the needle-Huber protective device of FIG. 15, with the Huber needle removed and a spring and shield of the device in a closed position.

FIG. 17 is a side elevational view of a portion of the needle-Huber protective device of FIG. 1, showing the device in a compressed position with the Huber-needle extending through the access hole of the base cap.

### DETAILED DESCRIPTION OF THE INVENTION

The present invention can be understood more readily by reference to the following detailed description, examples, drawings, and claims, and their previous and following description. However, before the present devices, systems, and/or methods are disclosed and described, it is to be understood that this invention is not limited to the specific devices, systems, and/or methods disclosed unless otherwise specified, as such can, of course, vary. It is also to be understood that the terminology used herein is for the purpose of describing particular aspects only and is not intended to be limiting.

As used in the specification and the appended claims, the singular forms “a,” “an” and “the” include plural referents unless the context clearly dictates otherwise. Thus, for example, reference to a “needle” can include two or more such needles unless the context indicates otherwise.

Ranges can be expressed herein as from “about” one particular value, and/or to “about” another particular value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint.

As used herein, the terms “optional” or “optionally” mean that the subsequently described event or circumstance can or cannot occur, and that the description includes instances where said event or circumstance occurs and instances where it does not.

Reference will now be made in detail to the present preferred embodiment(s) of the invention, examples of which are illustrated in the accompanying drawings. Wherever possible, the same reference numbers are used throughout the drawings to refer to the same or like parts.

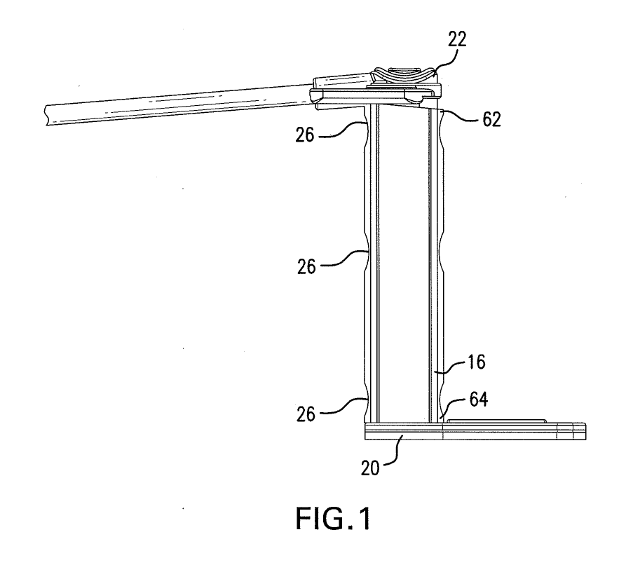

FIG. 1 of the accompanying drawings illustrates an embodiment of a needle protective device 10 and a needle assembly 22. In one aspect, the needle protective device can be comprised of a tubular member 16, a base cap 18, and an antimicrobial barrier 20. The needle assembly 22 can comprise a needle hub, wings 58, and an elongate needle 14 extending from the needle hub. In one aspect, the needle can be a Huber needle. In another aspect, the needle 14 can be a non-coring Huber needle.

In one aspect, the antimicrobial barrier 20 comprises a protective laminated dressing that provides passage of oxygen and/or other fluids at a wound site. In another aspect, the protective laminated dressing can be constructed on and/or formed from a fabric comprising metallic silver and/or silver oxide that are configured to release a sustained level of silver ions onto the wound site to provide an antimicrobial barrier at the wound site.

The tubular member 16 can have a longitudinal axis, a first end 62 and a second end 64. In one aspect, the first end of the tubular member can be configured for mounting on the needle hub, as will be described below. In another aspect, the second end of the tubular member can be configured for mounting the base cap 18 thereon, as will also be described below. In another aspect, the tubular member can be selectively axially movable between a first relaxed position and a second compressed position by urging the second end 64 of the tubular member substantially along the longitudinal axis of the tubular member 16 towards the first end 62. In this aspect, the tubular member can store resilient force when the tubular member is moved from the first relaxed position to the second compressed position.

The tubular member 16 can have a plurality of axial slits 24 formed thereon that extend axially over at least a portion of the tubular member. Optionally, the axial slits 24 can be diametrically opposed to each other and can have at least one notch 26 selectively formed along a portion of the edge of the axial slits, which allow portions of the tubular member to controllably bow outwardly when the tubular member is axially compressed.

In one aspect, the tubular member 16 can be formed from resilient polymeric materials which contain strings of non-stretching materials to prevent the elasticity of tubular member from setting. In another aspect, the tubular member can be formed from resilient polymeric materials which contains strings of non-stretching materials to provide a predetermined dimension between the base cap 18 and the needle assembly 22, before, during and after the activation process, as will be described more fully below.

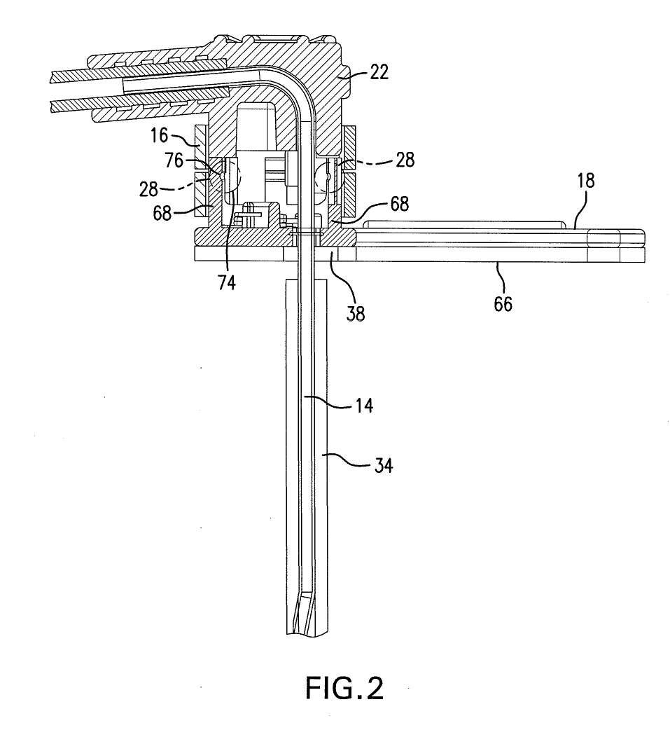

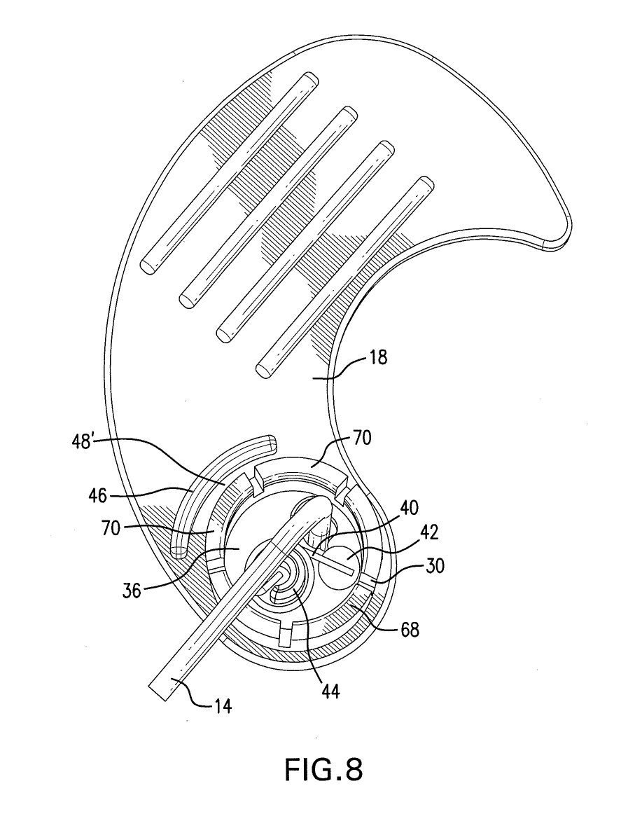

Referring to FIG. 2, in one aspect, the base cap can have a substantially planar single wing 66 having a distal surface that faces away from the tubular member 16 when the base cap 18 is mounted to the tubular member. In another aspect, the base cap can have a first circular protrusion 68 having a first diameter, the first protrusion extending in a direction generally away the distal surface of the wing 66 of the base cap. In another aspect, as illustrated in FIG. 8, a plurality of slots 30 can be formed in the first circular protrusion. In this aspect, the plurality of slots can extend from an end of the first circular protrusion 68 towards the distal surface of the wing 66 of the base cap 18 so that the first circular protrusion can comprise a plurality of tabs 70. In still another aspect, a groove 28 can be defined therein the first circular protrusion, the groove being substantially parallel to the distal surface of the wing of the base cap 18. As can be appreciated, it is contemplated that the size, shape, and/or the number of slots of the plurality of slots 30 can adjust or affect the flexibility of the tabs 70. In one aspect, the slots can assist in the rejection of the base cap 18 from molding tooling during the manufacturing process.

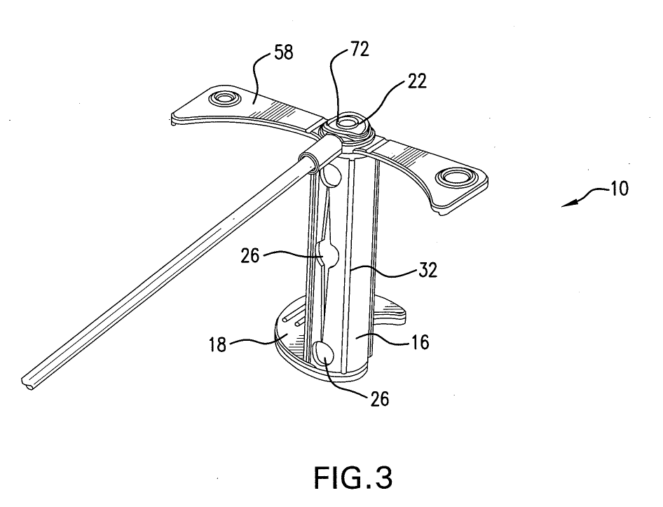

In one aspect, as illustrated in FIG. 3, the needle assembly 22 can have a proximal end 72 that faces away from the tubular member 16 when the needle assembly is mounted to the tubular member. In another aspect, the needle assembly can have a second circular protrusion 74 (illustrated in FIG. 2) having a second diameter, the second protrusion extending in a direction generally away from the proximal end 72 of the needle assembly. In another aspect, a plurality of slots can be formed in the second circular protrusion 74. In this aspect, the plurality of slots can extend from an end of the second circular protrusion of the needle assembly toward the proximal end of the needle assembly 22 so that the second circular protrusion can comprise a plurality of tabs. In another aspect, the second diameter of the second circular protrusion 74 of the needle assembly can be less than the first diameter of the first circular protrusion 68 of the base cap so that the second circular protrusion of the needle assembly can fit inside the first circular protrusion of the base cap 18. In still another aspect, a ring 76 can be formed on an outer diameter of the second circular protrusion 74 of the needle assembly, the ring being configured to matingly engage the groove 28 of the base cap 18.

In one aspect, when the base cap 18 is mounted to the second end 64 of the tubular member 16, the needle protective device 10 can be pre-activated by urging the base cap axially toward the needle assembly 22, thereby moving the tubular member to the second compressed position. In another aspect, the groove 28 of the base cap 18 can selectively matingly engage the ring 76 of the needle assembly 22 to create a temporary holding force that is greater than the stored resilient force within the tubular member 16. The temporary holding force can provide a holding mechanism between the base cap 18 and the needle assembly 22 to prevent the base cap from inadvertently moving away from the base cap. Optionally, the groove 28 and ring 76 holding force can be increased or decreased by changing number and/or the size of the groove and/or the ring, and/or by increasing the number, location, and/or the width of the slots 30 of the base cap 18 and/or the needle assembly 22.

In another aspect, the needle assembly 22 can comprise mis-alignment geometries that pre-skew the needle 14 so that in a pre-activated position, (i.e., when the tubular member is in the first, relaxed position) the needle is not co-axially aligned with the longitudinal axis of the tubular member 16 and/or the needle 14 is not co-axially aligned with a longitudinal axis of an access hole 38 of the base cap 18. Thus, when the needle protective device 10 is in the pre-activated position, the needle cannot inadvertently pass through the access hole 38 of the base cap because the tip of the needle 14 is not aligned with the access hole.

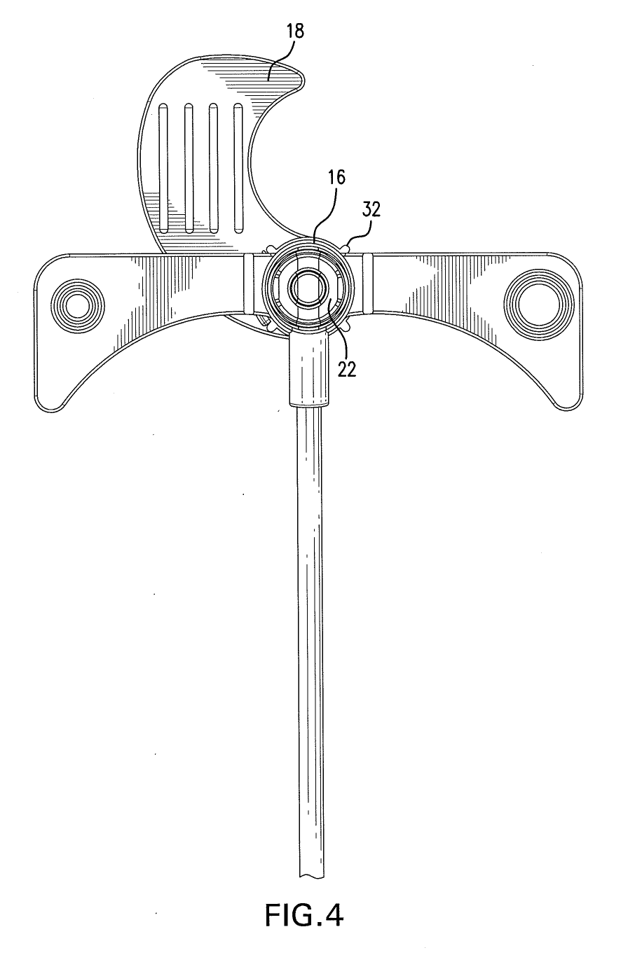

Referring to FIGS. 3 and 4, the tubular member 16 can be cylindrical in shape having a circular cross-sectional of substantially constant diameter and contain ribs 32 that support and or align the tubular member to the base cap 18 and needle assembly 22. It is contemplated, however that the tubular member 16 cross-sectional shape can be other shapes, such as, for example, and not meant to be limiting, square, rectangular, or oval.

The tubular member 16 can be formed from resilient polymeric materials, such as for example and not meant to be limiting, silicone rubber or PVC and can contain strings of non-stretching materials to prevent the elasticity of tubular members. In one aspect, the tubular member 16 can withstand gamma or other radiation for sterilization purposes, and can be stable up to a temperature of at least 200 degrees C. The tubular member 16 can also be ultraviolet resistant to a substantial extent. In yet another aspect, at least a portion of the tubular member 16 can be substantially transparent, however, it is contemplated that at least a portion of the tubular member 16 can be color coded to indicate, for example, needle dimensions or other needle properties. It is also contemplated that tubular member 16 can be formed by standard manufacturing processes, such as, for example and not meant to be limiting, extrusion or injection molding.

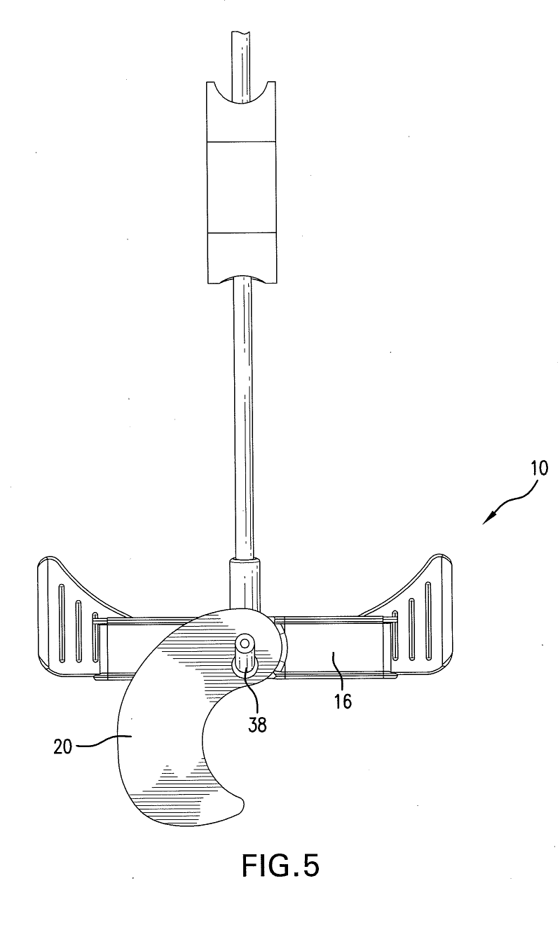

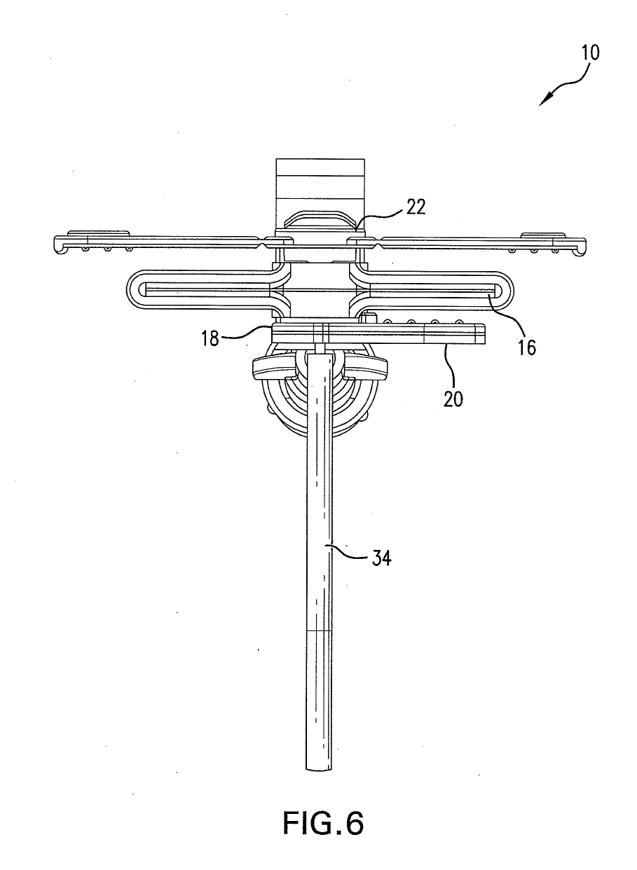

FIGS. 5 and 6 of the accompanying drawings illustrate embodiments of the protective device 10 showing the tubular member 16 in the compressed position. As illustrated, the needle protective device can further comprise a conventional flexible tube 34 configured to protect the needle 14 when the needle is in an extended position through the access hole 38 of the base cap 18. In use, when the tubular member 16 is moved from the first compressed position to the second relaxed position, the tubular member 16 releases a stored resilient force within the tubular member 16 and activates one of the safety feature within the needle protective device 10, described more fully below.

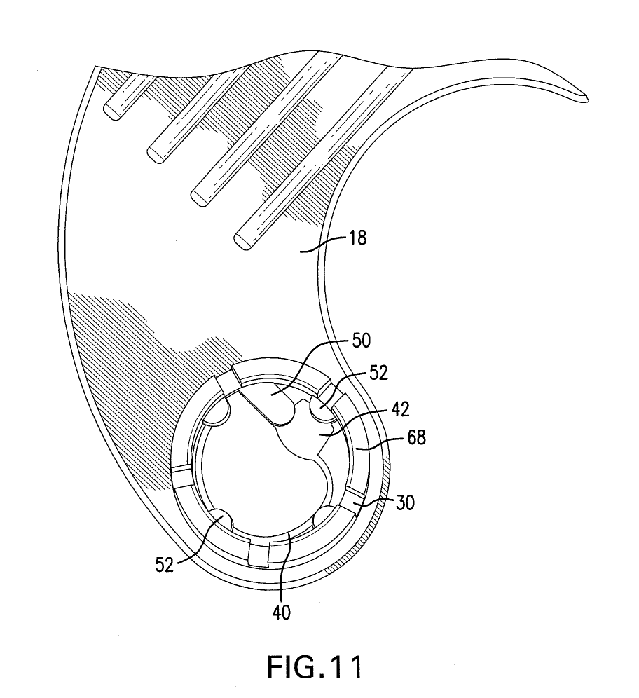

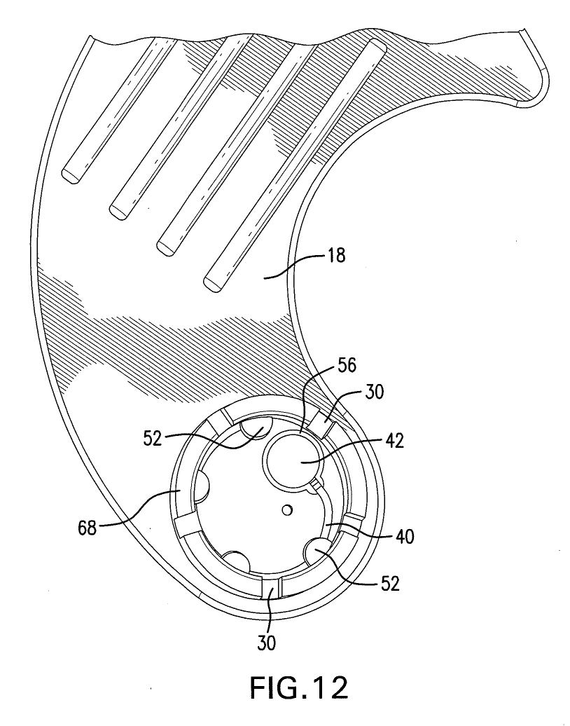

FIGS. 8, 9, 11 and 12 illustrate various embodiments of the base cap 18. In one aspect, the base cap can be formed from a rigid material and can define a chamber 36 having the access hole 38 through which the needle 14 of the device can be passed. In another aspect, the base cap can further comprise a spring 40 and a shield 42. In another aspect, the spring can be mounted and/or bonded around and/or within a spring support column 44. In still another aspect, the spring can be a pre-activated torsion spring.

In one aspect, the base cap can further comprise at least one stop ridge 60, at least one tab 52, and/or at least one tongue 50 that can prevent the spring from extending past a predetermined position. In this aspect, a portion of the spring 40 and/or the shield 42 can lock or rest against the at least one stop ridge 60, at least one tab 52, and/or at least one tongue 50 thereby preventing movement of the spring 40 beyond a predetermined position. In another aspect, the spring 40 can be formed from a predetermined material having a predetermined gauge, and/or number of coils to selectively increase or decrease the amount of resilient force stored within a compressed spring.

In one aspect, the spring and/or the shield can rest against the needle 14 when the needle is extended through the access hole. In another aspect, when the needle is retracted through the access hole 38, the spring 40 can urge the shield 42 to block the access hole. In use, the spring 40 can urge the shield 42 to cover the access hole 38 of the base cap when the needle 14 is not extended through the access hole. In another aspect, activation of the device 10 passively activates the shield, covering the access hole of the base cap providing an additional safety feature as well as a means to prevent reuse or reactivation of the device.

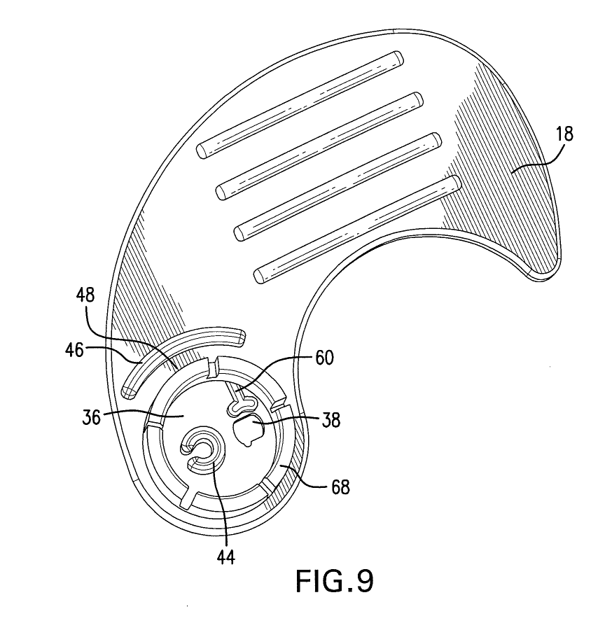

As illustrated in FIGS. 8 and 9, the base cap 18 can further comprise at least one channel ridge 46 configured to matingly engage the tubular member 16, in order to more readily align the base cap 18 with the tubular member 16 and/or the ribs 32 of the tubular member. This can, according to one aspect, provide manufacturing advantages and alignment advantages for the tubular member 16 in both compressed and decompressed positions. In still another aspect, the at least one channel ridge 46 can provide bonding locations, and/or bonding and/or gluing channels 48 configured to hold an adhesive, thereby improving manufacturability and/or quality of mated components.

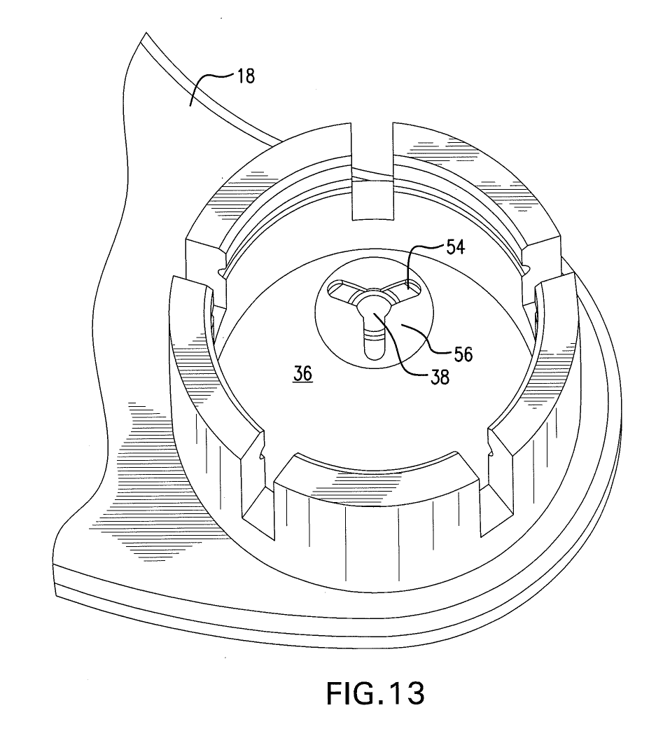

FIG. 13 illustrated another aspect of the needle protective device 10, wherein the base cap 18 further comprises at least one locking member 56 inserted or formed therein adjacent and/or aligned with the access hole 38. In another aspect, the at least one locking member can define at least one locking slot 54 configured to interact in a flexible one-way manner to created a closed position to prevent the needle 14 from exiting the chamber 36 of the base cap 18, thus preventing the reuse of the device 10. In this aspect, the at least one locking member 56 can provide tactile feedback through beveled and/or shaped surfaces to a user when the needle is in the process of being locked. Additionally, in this aspect, the at least one locking member 56 can support the needle 14 in the pre-activated position while guiding the needle through the access hole 38 during passive activation.

In another aspect, access to the access hole 38 can be restricted by a plurality of slotted and/or molded resilient elements within either a raised position, a domed position, and/or a flat position. In this aspect, through the activation process, the slotted and/or molded resilient elements can be compressed around the needle 14. In another aspect, when the needle tip is contained within the end base, the slotted and or molded flexible elements can collapse around and/or over the access hole 38 by means of the resilient force stored within the slotted and/or molded resilient elements materials, preventing the needle from exiting the base cap.

In order to pre-activate the device 10, a user must co-axially align the tip of the needle 14 with the access hole 38 of the base cap 18. According to one aspect, after pre-activation of the device, the safety features of the needle protective device can be passively activated by: i) the pre-activated spring 40 so that upon removal of the needle tip from the access hole 38, the shield 42 can block the access hole; and/or ii) the decompression of the plurality of slotted and/or molded resilient elements so that the tip of the needle 14 can be moved within the chamber 36 and away from the access hole 38 by means of the resilient force stored within the slotted and/or molded resilient elements.

In one embodiment, when the needle tip has been pre-aligned with the access hole 38 of the base cap 18, the needle protective device 10 can be activated during the clinical procedure by applying downward pressure to the single wing 66 of the base cap thus holding the base cap against the skin surface of the patient. Simultaneously, upward pressure can be applied to the wings 58 of the needle assembly 22 to overcome the temporary holding force of the mated ring 76 and groove 28. This can allow users of the device 10 to only expose one finger at the wound site (i.e., adjacent the base cap) throughout the activation and post clinical infusion procedures.

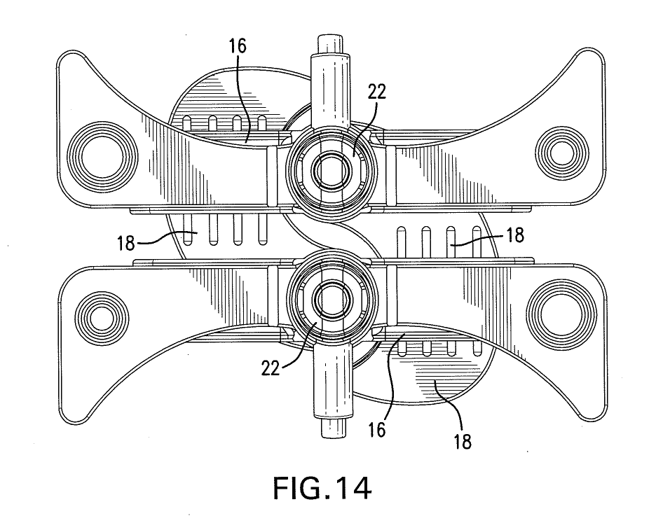

FIG. 14 illustrates two needle protective devices mounted adjacent each other. In one aspect, each base cap 18 can be shaped to mate with another base cap 18 to allow multiple infusion at one port site. In this aspect, the base cap 18 geometries can be selected to provide close center-to-center needle access hole 38 locations to reduce the overall footprint of the devices 1 on the skin of a patient. In another aspect, the base cap 18 geometries can interlock with one another provide a stable clinical platform on a port infusion site.

In use, in one aspect, the needle protective device 10 can be activated by a single finger of a user. In another aspect, the needle protective device of the current application can reduce the exposure and risk to a clinical provider by at least 10%, 20%, 25%, 30%, 40%, 50%, 60%, 70%, 75%, 80%, and 90% when compared to conventional two finger base wing needle protective devices.

### Example

The following example is put forth so as to provide those of ordinary skill in the art with a complete disclosure and description of how the device, systems, and/or methods described and claimed herein are made, and is intended to be purely exemplary and is not intended to limit the scope of what the inventors regard as their invention.

In an exemplary aspect, the needle protective device comprising a tubular member that can be constructed of resilient flexible material and can be mounted between a needle assembly and a base cap.

The tubular member can be compressed in the pre-activated position and held in a compressed position through force balancing utilizing a ring and groove and by activation extending at least a portion of a needle tip from the base cap. The tubular member can be a flexible tubular member selectively axially movable between a first compressed position and a second relaxed position such that, in operation, when the tubular member is moved from the first compressed position to the second relaxed position, the tubular member releases a stored resilient force within the tubular member. In operation, when the tubular member is moved from the first compressed position to a second relaxed position, the tubular member resilient force releases and can assist in the mis-alignment of the needle tip in relation to the access hole.

In another aspect, the flexible tubular member can have a plurality of axial slits formed thereon that extend axially over at least a portion of the tubular member. The axial slits can be diametrically opposed to each other and can have notches selectively formed along a portion of the edges of the axial slits, which allow portions of the tubular member to controllably bow outwardly when the tubular member is axially compressed.

Without limitation, it is contemplated that the tubular member can formed from resilient polymeric materials which contains strings of non-stretching materials to prevent the elasticity of tubular member from setting and/or to provide a predetermined dimension between the base cap and the needle assembly, before, during and after the activation process.

In a further aspect, the base cap defines a groove, and the needle assembly comprises a ring, wherein the ring and groove are configured to selectively, complementarily mate or otherwise engage each other. It is contemplated in this example, that the holding force of the engagement of the ring and groove is greater than the stored resilient force within the tubular member thereby providing a selective and temporary holding mechanism between the base cap and the needle assembly. In various aspects, the holding force can be selectively changeable by increasing or decreasing: i) sizing and surface area geometries of the ring and/or the groove; ii) by increasing the number, location, and/or width of slots formed therein the base cap and the needle assembly; iii) increasing or decreasing the geometries of axial slits that extend axially over at least a portion of the tubular member; iv) material selection of the elements; and v) and the interactions of these defined mechanisms.

In another aspect, the base cap can comprise at least one stop ridge inserted or formed therein and configured to restrict movement of the spring and/or the plate cover to a predetermined position. Optionally, the base cap has at least one locking member and/or slot inserted or formed therein that interact in a flexible, locking and/or keyed manner to allow a one-way directional open to closed position that locks the needle 14 within a chamber of the base cap. The at least one locking member can be configured to provide a tactile feedback to a user when the needle is retracted during the process of locking.

In another aspect, an access hole can be defined in the base cap that is mis-aligned and covered by a plurality of slots with at least one locking member in a raised, domed, and/or flat position. Thus, in operation and during the activation process, at least portions of the at least one locking member and/or the plurality of slots frictionally engage the needle 14, and when the tip of the needle is in a chamber of the base, restricts access of the needle to the access hole.

In a further aspect, the base cap can comprises a pre-activated torsion spring and a shield that is supported on a post of the base cap. Here, the spring can be formed from a material having a predetermined gauge. As one skilled in the art will appreciate, the spring can be conventionally formed with a predetermined number of coils to increase or decrease the amount of resilient force stored within the spring.

Optionally, to improve manufacturability and/or quality of mated components, the base cap can comprise or define at least one channel that is configured to provide bonding locations and/or bonding and/or gluing channels that hold adhesive within the at least one channel. In a further aspect, the base cap can comprise or define at least one channel ridge configured to matingly engage the tubular member and/or ribs of the tubular members to ease alignment of the base cap to the tubular member.

In another aspect, at least a portion of the base cap can comprise a protective laminated dressing that provides passage of oxygen and/or other fluids at a wound site. In various non-limiting aspects, at least a portion of the dressing can be formed from a fabric comprising metallic silver and/or silver oxides that release a desired and/or sustained level of silver ions to the wound site thereby providing an antimicrobial barrier at the wound site.

In yet another aspect, the base cap can be shaped to complementarily mate with a second needle protective device to allow multiple infusions at one port site. In this aspect, the shape of the base cap can be configured to reduce the minimum center-to-center needle positions between the first and second needle protective devices, which reduces the overall footprint on a port infusion site of the patient. Additionally and optionally, it is contemplated that the base caps of the first and second needle protective devices can be configured to selectively inter-lock with one another to provide a stable clinical platform on a port infusion site.

In another aspect, the needle 14 is configured to rest against a spring with a shield that is mounted and/or bonded to a spring support column such that, in operation, when the needle passes through the access hole the needle activates the spring and when the needle 14 is retracted through the access hole, the shield blocks the access hole.

In operation, it is contemplated that the needle assembly is configured to pre-skew the needle into a non-auxiliary geometry so that the needle, when in a pre-activated position, is not co-axially aligned with a longitudinal axis of the tubular member and/or is not co-axially aligned with a longitudinal axis of the base cap access hole.

In operation, it is contemplated that the device can be activated using a single finger of a user. The device can reduce the exposure and risk to a clinical provider by at least 10%, 15%, 20%, 25%, 30%, 35%, 40%, 45%, 50%, 55%, 60%, 65%, 70%, 75%, 80%, 85%, or 90%, when compared to conventional needle protective devices.

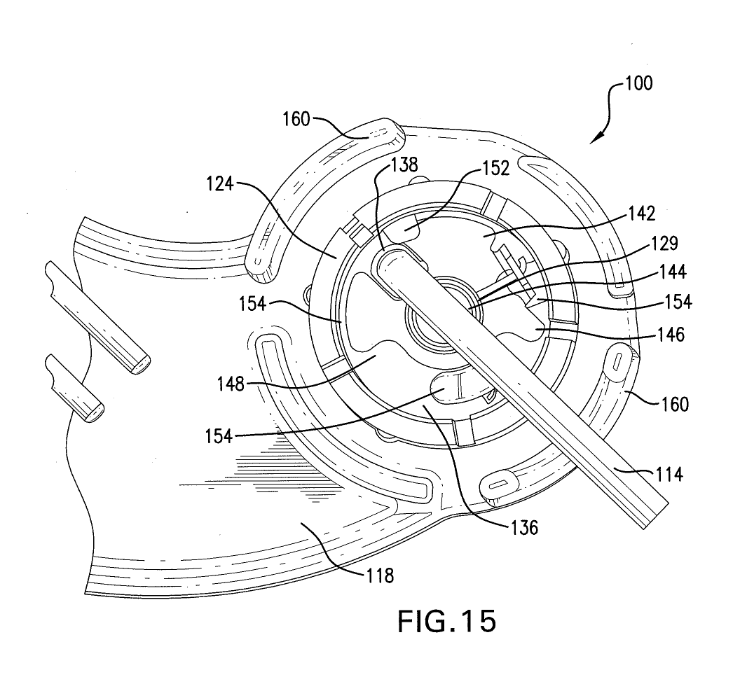

FIG. 15 of the accompanying drawings illustrates an embodiment of a needle-Huber protective device 100 for a needle assembly having a needle 114. In one aspect, the needle can be a Huber needle. In another aspect, the needle can be a non-coring Huber needle.

In one aspect, the needle-Huber protective device can comprise at least one of a base cap 118, a tubular member (not shown in FIG. 15), a spring 140, and a shield 142. In one aspect, the base cap 118 can be formed from a rigid material and has an outer wall 124 defining a chamber 136 configured to isolate the needle tip, as described more fully below. In another aspect, the base cap can have a first end and a second end, wherein the first end of the base cap is fixedly mounted on the tubular member and the second end of the base cap has a bottom surface 150. In another aspect, an access hole 138 can be defined in the bottom surface of the base cap through which a user of the device can selectively pass a tip of a needle 114, as illustrated in FIG. 15. As will be described more fully below, the tip of the needle can move outwardly distally to the second end of the base cap 118 as the needle assembly is moved from the pre-activated condition to the extended activated condition. In still another aspect, the base cap comprises at least one stop tab 152, 154.

In one aspect, the spring 140 and/or shield 142 can be mounted and/or bonded to a portion of the base cap 118, such as spring support column 144, and a portion of the spring can be mounted to the shield 142. For example and without limitation, the spring can be a torsion spring or the like. In another aspect, the spring 140 can be rotatable or otherwise movable about and between a compressed position, in which potential energy is stored in the spring, and a relaxed position, in which at least a portion of the potential energy stored in the spring is released. In still another aspect, the spring 140 can be formed with wire having a gauge and number of coils so that a predetermined amount of resilient force can be stored within the spring. It is contemplated that the amount of resilient force desired can be calculated based on human factor demands and/or the amount of force required to move and maintain the shield in the closed position.

In one aspect, the shield 142 can be a rotatable shield configured to selectively restrict or block access to the access hole 138 of the base cap 118. In another aspect, a portion 146 of the shield can be configured to selectively restrict movement of the shield within the base cap, or, optionally, block access to the access hole of the base cap. In still another aspect, the shield 142 can be coupled to a portion of the spring 140 so that when the spring moves between the compressed and the relaxed position, the shield is moved between an open position, in which passage through the access hole 138 is not restricted, thereby allowing for the passage of the needle through the access hole, and a closed position, in which access to or passage through the access hole is restricted or otherwise blocked. In yet another aspect, at least one notch 148 can be defined along the perimeter of the shield, at least a portion of the at least one notch being configured to engage portions of the at least one stop tab 152, 154 of the base cap.

In another aspect, the at least one stop tab 152, 154 can be formed in the base cap 118 to restrain the shield 142 so that the spring 140 is restrained to movement about and between the compressed and relaxed positions. In still another aspect, the at least one stop tab can be positioned so that the spring and/or the shield are positioned or locked in a desired position against the at least one stop tab. For example, a portion of a notch of the at least one notch 148 can contact a stop tab, thereby preventing further movement of the shield in the direction of the stop tab. In another aspect, a top surface of the at least one stop tab 152, 154 can be sloped (at an acute angle relative to the shield). In this aspect, the shield can rotate and slide over the sloped top surface of the stop tab in a first direction, while being preventing from rotating in an opposed, second direction.

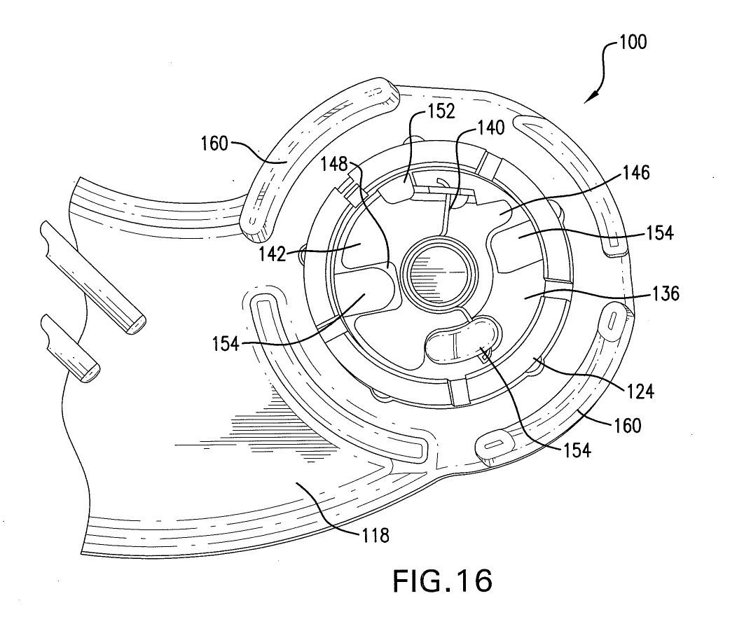

As illustrated in FIG. 16, the at least one stop tab 152, 154 can be a radial stop tab 152 that extends radially into the chamber 136 of the base cap from the base cap outer wall 124, or an axial stop tab 154 that extends longitudinally into the chamber from the lower wall of the base cap 118. The base cap can have at least one holding tab formed therein or molded thereto that is configured to ensure that the spring 140 and/or shield 142 rests in a closed or locked position and/or an open or unlocked position. In operation, the at least one stop tab 152, 154 and/or the at least one holding tab can prevent the spring and/or the shield from upward-lift movement and/or tampering once the shield has been installed into the base cap of a finished device 100.

Prior to use of the device, in one aspect, the needle hub and a wing member of a needle assembly 122 (as described above) can align the needle 114 through auxiliary geometries of the resilient or hinged tubular member (not illustrated) so that in the pre-activated position, a longitudinal axis of the needle is substantially parallel to a longitudinal axis of the tubular member, and/or that the needle is substantially co-axially aligned with the access hole 138 of the base cap 118. Thus, in the pre-activated condition, in one aspect, at least the tip of the needle 114 can be aligned to pass through the access hole 138.

As illustrated in FIG. 15, in a pre-activated condition, at least the tip of the needle 114 can be positioned therein the chamber 136. In this condition, the spring 140 is in the compressed position and the shield 142 of the base cap 118 can rest against the needle 114. The needle tip can be inserted into a patient and medication can be injected into the patient through the needle. The needle can continue to contact the shield 142 to prevent movement of the shield.

After injection of the medication, the tip of the needle 114 can be removed from the patient and withdrawn through the access hole 138 into the chamber 136 of the base cap 118. In the chamber, the needle tip can be positioned in a plane that is above the spring 140 and shield 142 of the base cap 118. Because the needle tip is positioned above the spring and shield, the needle 114 does not restrain movement of the shield, and the energy stored in the spring can cause the shield to move from the compressed position to the relaxed position. As the spring 140 moves from the compressed position to the relaxed position, the shield 142 moves from the open position to the closed position, and at least a portion of the shield prevents access to the access hole of the base cap. Thus, when the needle tip is withdrawn inwardly proximally to the chamber of the second end of the base cap as the needle assembly is moved from the extended activated condition to the withdrawn condition, the access hole can automatically be covered by the pre-activated shield 142. As mentioned above, the at least one stop tab 152, 154 can be formed or positioned in the base cap 118 to prevent movement of the shield beyond the desired closed and/or locked position. As mentioned above, it is contemplated that the at least one stop tab 152 can be formed or positioned in the base cap 118 to prevent vertical movement of the shield beyond the desired closed and/or locked position. In another aspect, when the device is activated (i.e., when the tip of the needle is removed from the patient and withdrawn through the access hole 138) the device can provides a tactile feedback to the user during the process of moving the shield 142 to the closed position so that the user can feel that the device is locked.

FIG. 16 illustrates an embodiment of the base cap 118 showing the spring 140 and the shield 142 after the needle 114 (not shown in FIG. 16) has been moved to a position above the spring and shield of the device (i.e., the device 100 is in a post-activated condition). In this position, the access hole 138 has been covered by the shield 142 and is not visible. The spring 140 continually exerts a force to the shield and against the at least one stop tab 152, 154, thereby preventing the shield from moving away from the closed position, and thus, prevents reuse of the needle 114. Further, because the shield remains in the closed position, the needle tip can be randomly positioned at any location within the base cap 118 and cannot inadvertently be removed from the base cap.

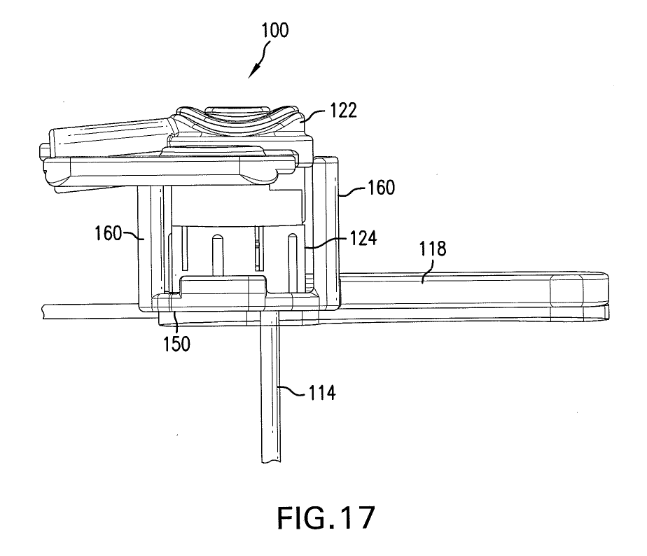

As illustrated in FIG. 17, a needle assembly 122 and base cap 118 are shown in a compressed pre-activated position (the tubular members are not illustrated). In one aspect, the base cap 118 can further comprise at least one secondary ridge 160 or wall that can act as a secondary barrier against needle 114 withdrawal from the base cap 118 and needle sticks during the activation process. In another aspect, the secondary ridge can be substantially perpendicular relative to the bottom surface 150 of the base cap. In still another aspect, the secondary ridge can have a height greater than the height of the outer wall 124 of the base cap 118. In another aspect, the secondary ridge 160 can have a height about two times the height of the base cap outer wall 124. In a further aspect, the secondary ridge can have a height less than the height of the primary wall. In one aspect, the configuration of the secondary ridge can help ensure optimal alignment of the base cap 118 to the tubular member during manufacturing for both the compressed and decompressed positions of the tubular member.

In another aspect, the base cap 118 of the needle-Huber protective device 100 can comprise at least one secondary vertical ridge 160 that is shaped to align and/or position the tubular or hinged member in a desired position relative to the base cap. In one aspect, the secondary vertical ridge can be configured to complementarily mate to tubular members so that the Huber needle hub and wing component members can be optimally aligned relative to the base cap 118. In another aspect, the at least one secondary vertical ridge 160 of the base cap can engage the tubular member and/or ribs of the tubular member, thereby providing alignment and manufacturing advantages when the tubular member is in both the compressed and a relaxed position. In still another aspect, the at least one secondary vertical ridge can have a predetermined height such that the at least one secondary vertical ridge 160 can act as a secondary barrier against needle 114 withdrawal from the base cap 118 and needle sticks during the activation process.

Although several embodiments of the invention have been disclosed in the foregoing specification, it is understood by those skilled in the art that many modifications and other embodiments of the invention will come to mind to which the invention pertains, having the benefit of the teaching presented in the foregoing description and associated drawings. It is therefore understood that the invention is not limited to the specific embodiments disclosed herein, and that many modifications and other embodiments of the invention are intended to be included within the scope of the invention. Moreover, although specific terms are employed herein, they are used only in a generic and descriptive sense, and not for the purposes of limiting the described invention.

What is claimed is:

1. A needle protective device for a needle assembly having a needle hub and a needle projecting from the hub, the needle having a needle tip, the needle protective device comprising:
a tubular member of resilient flexible material which extends about at least a portion of the needle, the tubular member having a longitudinal axis, a first end and a second end, wherein the first end of the tubular member is mounted on the needle hub, wherein the tubular member is selectively axially movable between a tube relaxed position and a tube compressed position, and wherein the tubular member stores resilient force when the tubular member is moved from the tube relaxed position, in which the needle assembly is in a pre-activated condition and a withdrawn condition, to the tube compressed position, in which the needle assembly is an extended activated condition; and
a base cap having a first end and a second end, wherein the first end of the base cap is fixedly mounted on the second end of the tubular member, wherein the base cap has an outer wall defining a chamber, wherein the second end of the base cap defines an access hole in communication with the chamber through which a user of the device can selectively pass a tip of the needle outwardly distally to the second end of the base cap as the needle assembly is moved from the pre-activated condition to the extended activated condition, wherein at least the tip of the needle is positioned therein the chamber when the needle assembly is in the pre-activated condition;
a spring coupled to a portion of the base cap, wherein the spring is movable about and between a spring compressed position and a spring relaxed position; and
a shield configured to restrict access to the access hole, wherein the shield is coupled to a portion of the spring, wherein as the spring moves between the spring compressed position to the spring relaxed position, the shield is moved between an open position, in which the needle can pass through the access hole, and a closed position, in which access to the access hole by the needle is restricted, and wherein the spring is configured to automatically move between the spring compressed position to the spring relaxed position upon the withdrawal of the needle tip inwardly proximally to the chamber of the second end of the base cap as the needle assembly is moved from the extended activated condition to the withdrawn condition.

2. The needle protective device of claim 1, wherein the base cap comprises at least one stop tab therein, the at least one stop tab configured to restrain the shield so that the spring is restrained to movement about and between the spring compressed and spring relaxed positions.

3. The needle protective device of claim 2, wherein at least one notch is defined along the perimeter of the shield, the at least one notch configured to engage portions of the at least one stop tab of the base cap.

4. The needle protective device of claim 3, wherein the at least one stop tab is positioned such that the shield is locked in a desired position against the at least one stop tab.

5. The needle protective device of claim 4, wherein a top surface of the at least one stop tab is positioned at an acute angle relative to the shield such that the shield can slide over the top surface of the at least one stop tab in a first direction, while being preventing from moving in an opposed, second direction.

6. The needle protective device of claim 2, wherein the at least one stop tab is a radial tab extending radially into the chamber from the outer wall of the base cap.

7. The needle protective device of claim 2, wherein the base cap further comprises at least one secondary ridge configured to restrict needle withdrawal of the tip of the needle from the base cap.

8. The needle protective device of claim 7, wherein the at least one secondary ridge has a height greater than the outer wall of the base cap.

9. The needle protective device of claim 1, wherein, in the pre-activated condition, at least the tip of the needle is aligned to pass through the access hole.

10. The needle protective device of claim 9, wherein, in the withdrawn condition, the tip of the needle is positioned in a plane above the spring and shield.

11. The needle protective device of claim 10, wherein, in the withdrawn condition, access to the access hole by the needle is restricted by the shield.

11. The needle protective device of claim 1, wherein the spring is a torsion spring.

12. A needle protective device comprising:
a syringe comprising:
a hollow barrel having an inner diameter;
an end wall closing the barrel at a forward end of the syringe;
an open rear end of the syringe;
a piston means in reciprocable sealing engagement with the interior of the barrel defining a first chamber in said barrel for selectively containing fluid;
a needle hub mounted on the end wall defining an interior passage; and
an aperture in the end wall communicating the interior passage of the needle hub with the first chamber;
a needle coupled to and projecting outwardly from the needle hub;
a tubular member of resilient flexible material which extends about at least a portion of the needle, the tubular member having a longitudinal axis, a first end and a second end, wherein the first end of the tubular member is mounted on the needle hub, wherein the tubular member is selectively axially movable between a tube relaxed position and a tube compressed position, and wherein the tubular member stores resilient force when the tubular member is moved from the tube relaxed position, in which the syringe is in a pre-activated condition and a withdrawn condition, to the tube compressed position, in which the syringe is an extended activated condition; and
a base cap having a first end and a second end, wherein the first end of the base cap is fixedly mounted on the second end of the tubular member, wherein the base cap has an outer wall defining a chamber for protecting a tip of the needle, wherein the second end of the base cap defines an access hole in communication with the chamber through which a user of the device can selectively pass a tip of the needle outwardly distally to the second end of the base cap as the syringe is moved from the pre-activated condition to the extended activated condition, wherein at least the tip of the needle is positioned therein the chamber when the syringe is in the pre-activated condition;
a spring coupled to a portion of the base cap, wherein the spring is movable about and between a spring compressed position and a spring relaxed position; and
a shield configured to restrict access to the access hole, wherein the shield is coupled to a portion of the spring, wherein as the spring moves between the spring compressed position and the spring relaxed position, the shield is moved between an open position, in which the needle can pass through the access hole, and a closed position, in which access to the access hole by the needle is restricted, and wherein the spring is configured to automatically move between the spring compressed position to the spring relaxed position upon the withdrawal of the needle tip inwardly proximally to the chamber of the second end of the base cap as the syringe is moved from the extended activated condition to the withdrawn condition.

13. A method for reducing inadvertent needle sticks in infusion clinical situations comprising:
providing a needle protective device for a needle assembly having a needle hub and a needle projecting from the hub, the needle having a needle tip, the needle protective device comprising:
a tubular member of resilient flexible material which extends about at least a portion of the needle, the tubular member having a longitudinal axis, a first end and a second end, wherein the first end of the tubular member is mounted on the needle hub, wherein the tubular member is selectively axially movable between a tube relaxed position, in which the syringe is in a pre-activated condition and a withdrawn condition, to the tube compressed position, in which the syringe is an extended activated condition, and wherein the tubular member stores resilient force when the tubular member is moved from the tube relaxed position to the tube compressed position; and
a base cap having a first end and a second end, wherein the first end of the base cap is fixedly mounted on the second end of the tubular member, wherein the base cap has an outer wall defining a chamber for protecting a tip of the needle, wherein the second end of the base cap defines an access hole in communication with the chamber through which a user of the device can selectively pass a tip of the needle outwardly distally to the second end of the base cap as the needle assembly is moved from a pre-activated condition to an extended activated condition, wherein at least the tip of the needle is positioned therein the chamber when the needle assembly is in the pre-activated condition;
a spring coupled to a portion of the base cap, wherein the spring is movable about and between a spring compressed position and a spring relaxed position; and
a shield configured to restrict access to the access hole, wherein the shield is coupled to a portion of the spring, and wherein as the spring moves between the spring compressed position and the spring relaxed position, the shield is moved between an open position, in which the needle can pass through the access hole, and a closed position, in which access to the access hole by the needle is restricted and wherein the spring is configured to automatically move between the spring compressed position to the spring relaxed position upon the withdrawal of the needle tip inwardly proximally to the chamber of the second end of the base cap as the needle assembly is moved from the extended activated condition to a withdrawn condition;
inserting at least the tip of the needle of the needle assembly that is positioned in the extended activated condition into the patient;
injecting medication through the needle into the patient;
removing the needle from the patient; and
withdrawing the needle tip through the chamber of the base cap to the withdrawn condition to allow for the activation of the shield.
